(19) United States
(12) Patent Application Publication
(10) Pub. No.: US 2014/0240713 A1
(43) Pub. Date: Aug. 28, 2014

(54) APPARATUSES AND METHODS FOR IMAGING INSIDE A VESSEL

(71) Applicant: VOLCANO CORPORATION, San Diego, CA (US)

(72) Inventor: Nathaniel J. Kemp, Concord, MA (US)

(73) Assignee: VOLCANO CORPORATION, San Diego, CA (US)

(21) Appl. No.: 14/136,169

(22) Filed: Dec. 20, 2013

Related U.S. Application Data
(60) Provisional application No. 61/745,216, filed on Dec. 21, 2012.

(51) Int. Cl. A61B 5/00; G01B 11/24

(52) U.S. Cl. CPC A61B 5/0073; G01B 11/24; G01B 11/2441
USPC 356/479; 356/601

(57) ABSTRACT

The invention generally relates to apparatuses and methods for imaging inside a vessel. In certain aspects, the invention provides an apparatus that includes an imaging device configured to image an inside of a vessel, and a sensing device configured to detect a conformational shape of the apparatus.

### CROSS-REFERENCE TO RELATED APPLICATIONS

This application claims the benefit of, and priority to, U.S. Provisional Application Ser. No. 61/745,216, filed Dec. 21, 2012, the contents of which are incorporated by reference herein in its entirety.

### FIELD OF THE INVENTION

The invention generally relates to apparatuses and methods for imaging inside a vessel.

### BACKGROUND

Biomedical imaging technology is rapidly advancing. For example, magnetic resonance imaging (MRI), X-ray computed tomography, and confocal microscopy are all in widespread research and clinical use, and have resulted in fundamental and dramatic improvements in health care. However, there are many situations in which existing biomedical imaging technologies are not adequate. In those situations, such imaging technology does not provide a physician with the required diagnostic information, and the physician must resort to other invasive examinations, such as biopsy and histopathologic examination, in order to obtain the required diagnostic information. Such examinations are potentially harmful, time consuming, and costly. Furthermore, there are many situations in which conventional excisional biopsy is not possible. Coronary artery disease, a leading cause of morbidity and mortality, is one important example of a disease in which conventional diagnostic excisional biopsy cannot be performed.

Imaging technologies have been developed that addresses those concerns. For example, Intravascular Ultrasound (IVUS) is an important interventional diagnostic procedure for imaging atherosclerosis and other vessel diseases and defects. In the procedure, an IVUS catheter is threaded over a guidewire into a blood vessel, and images are acquired of the atherosclerotic plaque and surrounding area using ultrasonic echoes. That information is much more descriptive than information from other imaging techniques, such as angiography, which shows only a two-dimensional shadow of a vessel lumen.

Development of depth-resolved light reflection or Optical Coherence Tomography (OCT) provides a high resolution imaging technique that also addresses those concerns. OCT is an imaging technique that captures micrometer-resolution, three-dimensional images from within optical scattering media (e.g., biological tissue). OCT uses a narrow line width tunable laser source or a superluminescent diode source to emit light over a broad bandwidth to make in situ tomographic images with axial resolution of less than 10 μm and tissue penetration of 2-3 mm. OCT provides tissue morphology imagery at much higher resolution than other imaging modalities such as MRI. Further, with such high resolution, OCT can provide detailed images of a pathologic specimen without cutting or disturbing the tissue.

A problem with IVUS and OCT is that those technologies only display a three dimensional image of a vessel using two dimensional linear projections. Accordingly, the images produced by IVUS and OCT do not account for distortions due to catheter eccentricity or an angle of the catheter inside a lumen.

### SUMMARY

The invention combines imaging devices with three dimensional shape sensing devices in order to produce more accurate images of an inside of a vessel. With apparatuses and methods of the invention, the imaging device obtains image data of an inside of the vessel. The sensing device detects a conformational shape of the imaging device. The sensing data is used in combination with the image data to construct a three dimensional image of an inside of the vessel. Images produced by methods and apparatuses of the invention are more accurate than previously constructed images from intravascular devices because images of the invention account for distortions, such as catheter eccentricity or an angle of the catheter inside a lumen.

The imaging device and the sensing device may be combined or separate devices. In certain embodiments, the imaging device and the sensing device are combined in a single apparatus. Generally, the single apparatus includes a hollow body configured to fit within a lumen of a vessel. In that embodiment, the imaging device and the sensing device are each at least partially disposed within the hollow body.

Any imaging device known in the art may be used with apparatuses and methods of the invention. Exemplary imaging devices include intravascular ultrasound (IVUS) devices and optical coherence tomography (OCT) devices. In certain embodiments, the imaging device is an OCT device. Typically, the OCT device includes a light source and at least optical fiber. In embodiments, in which the imaging device and the sensing device are combined in a single apparatus, the sensing device may employ the light source and the optical fiber of the OCT device to detect the conformational shape of the imaging device. This embodiment is advantageous because components of the OCT device are shared with the shape sensing device, which saves costs, space, and other valuable resources. In such embodiments, the sensing device generally includes at least one Fiber Bragg Grating strain sensor and works by interferometic interrogation. However, the sensing device is not limited to those components and that configuration and may be any sensing device that is configured to detect a conformational shape of the imaging device.

### BRIEF DESCRIPTION OF THE DRAWINGS

FIG. 1 shows an exemplary three dimensional rendering of a lumen vessel border using 2 dimensional linear projections from a prior art imaging device.

FIG. 2 shows an exemplary image using the image data from FIG. 1 while also integrating shape sensing data.

FIG. 3A is a schematic representation of a fiber optic position and shape sensing device of the present invention having two fiber cores.

FIG. 3A-1 is an enlarged view of a portion of the multicore optical fiber of FIG. 3A.

FIG. 3B is a schematic representation of a preferred embodiment of the fiber optic position and shape sensing device of the present invention having two fiber cores and a broadband reference reflector.

FIG. 3B-1 is an enlarged view of a portion of the multicore optical fiber of FIG. 3B.

FIG. 4A is a schematic representation of a fiber optic position and shape sensing device of the present invention having three fiber cores.

FIG. 4A-1 is an enlarged view of a portion of the multicore optical fiber of FIG. 4A.

FIG. 4B is a schematic representation of a preferred embodiment of the fiber optic position and shape sensing device of the present invention having three fiber cores and a broadband reference reflector.

FIG. 4B-1 is an enlarged view of a portion of the multicore optical fiber of FIG. 4B.

FIG. 5 depicts a preferred embodiment where the optical fiber means is three single core optical fibers.

FIG. 5-1 is an enlarged view of the three single core optical fibers of the optical fiber means of FIG. 5.

FIG. 6 is a schematic representation of an optical arrangement for the fiber optic position and shape sensing device.

FIG. 6-1 is an enlarged view of a portion of the multicore optical fiber of FIG. 6.

FIG. 7 depicts a sensor frame.

FIG. 8 is a bend parameter schematic.

FIG. 9 depicts the bend geometry.

FIG. 10 depicts geometry of a fiber cross-section.

FIG. 11 illustrates bend plane geometry.

FIG. 12 illustrates an example of an apparatus that combines an OCT device and a sensing device.

### DETAILED DESCRIPTION

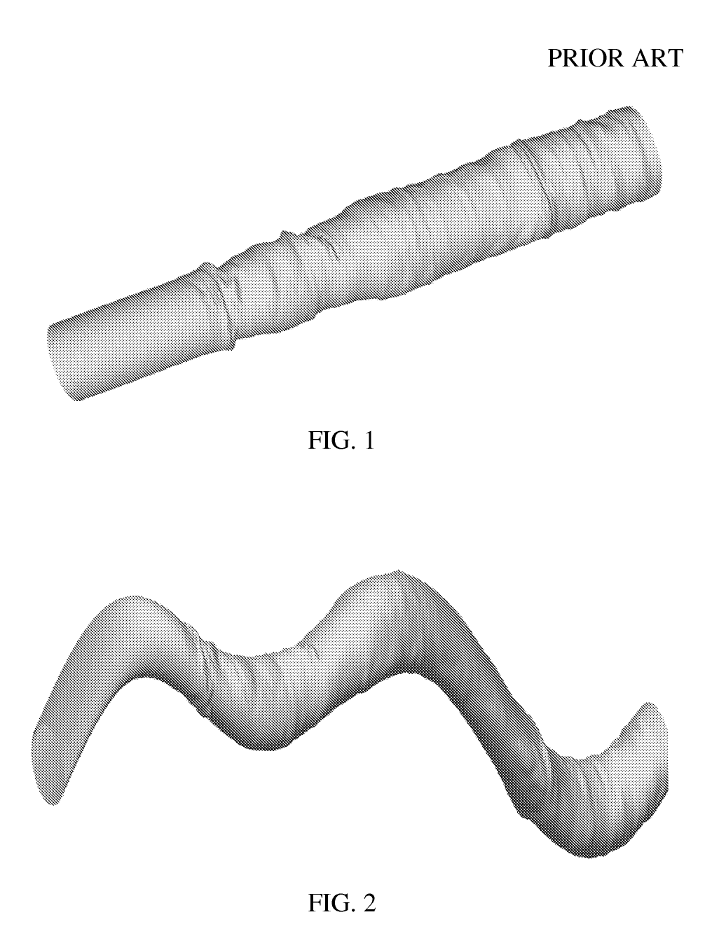

A typical problem with intravascular imaging devices is that those devices only display a three dimensional image of a vessel using two dimensional linear projections. FIG. 1 shows an exemplary prior art three dimensional rendering of a lumen vessel border using two dimensional linear projections from a prior art imaging device. That prior art image does not account for distortions due to catheter eccentricity or an angle of the catheter inside a lumen.

The invention combines imaging devices with three dimensional shape sensing devices in order to produce more accurate images of an inside of a vessel. FIG. 2 shows an exemplary image using the image data from FIG. 1 while also integrating shape sensing data. The three dimensional image is reconstructed based on the position (e.g., shape sensing data) of the catheter inside the vessel. The sensing data is used in combination with the image data to construct a three dimensional image of an inside of the vessel. Images produced by methods and apparatuses of the invention are more accurate that previously constructed IVUS and OCT images (See FIG. 1) because images of the invention account for distortions, such as catheter eccentricity or an angle of the catheter inside a lumen.

### Imaging Device

Any imaging device known in the art may be used with apparatuses and methods of the invention. Exemplary imaging devices include optical coherence tomography (OCT), spectroscopic devices, (including fluorescence, absorption, scattering, and Raman spectroscopies), intravascular ultrasound (IVUS), Forward-Looking IVUS (FLIVUS), high intensity focused ultrasound (HIFU), radiofrequency, optical light-based imaging, magnetic resonance, radiography, nuclear imaging, photoacoustic imaging, electrical impedance tomography, elastography, pressure sensing wires, intracardiac echocardiography (ICE), forward looking ICE and orthopedic, spinal imaging and neurological imaging, image guided therapeutic devices or therapeutic delivery devices, diagnostic delivery devices, and the like.

In certain embodiments, the imaging device is an OCT device. OCT systems and methods are generally described in Castella et al. (U.S. Pat. No. 8,108,030), Milner et al. (U.S. Patent Application Publication No. 2011/0152771), Condit et al. (U.S. Patent Application Publication No. 2010/0220334), Castella et al. (U.S. Patent Application Publication No. 2009/0043191), Milner et al. (U.S. Patent Application Publication No. 2008/0291463), and Kemp, (U.S. Patent Application Publication No. 2008/0180683), the content of each of which is incorporated by reference in its entirety. Additional description of OCT systems and methods is described in Kemp (U.S. Pat. No. 8,049,900), Kemp (U.S. Pat. No. 7,929,148), Milner (U.S. Pat. No. 7,853,316), Feldman et al. (U.S. Pat. No. 7,711,413), Kemp et al., U.S. Patent Application Publication No. 2012/0224751), Milner et al. (U.S. Patent Application Publication No. 2012/0136259), Kemp et al., (U.S. Patent Application Publication No. 2012/0013914), Milner et al. (U.S. Patent Application Publication No. 2011/0152771), and Kemp et al. (U.S. Patent Application Publication No. 2009/0046295), the content of each of which is incorporated by reference in its entirety.

OCT systems of the invention include a light source. The light source may be any light source generally used with OCT. Exemplary light sources include a narrow line width tunable laser source or a superluminescent diode source. Examples of narrow line width tunable laser sources include, but are not limited to, lasers having a Bragg diffraction grating or a deformable membrane, lasers having a spectral dispersion component (e.g., a prism), or Fabry-Pérot based tuning laser.

OCT systems of the invention also include an interferometer. The interferometer may be any interferometer generally used with OCT. Typically, the interferometer will have a differential beam path for the light or a common beam path for the light. In either case, the interferometer is operably coupled to the light source. In a differential beam path layout, light from a broad band light source or tunable laser source is input into an interferometer with a portion of light directed to a sample and the other portion directed to a reference surface. A distal end of an optical fiber is interfaced with a catheter for interrogation of the target tissue during a catheterization procedure. The reflected light from the tissue is recombined with the signal from the reference surface forming interference fringes (measured by a photovoltaic detector) allowing precise depth-resolved imaging of the target tissue on a micron scale. Exemplary differential beam path interferometers are Mach-Zehnder interferometers and Michelson interferometers. Differential beam path interferometers are further described for example in Feldman et al. (U.S. Pat. No. 7,783,337) and Tearney et al. (U.S. Pat. Nos. 6,134,003 and 6,421,164), the content of each of which is incorporated by reference herein in its entirety.

The differential beam path optical layout of the interferometer includes a sample arm and a reference arm. The sample arm is configured to accommodate and couple to a catheter. The differential beam path optical layout also includes optical circulators to. The circulators facilitate transmission of the emitted light in a particular direction. Circulators and their use in OCT systems are further described for example in B. Bouma et al. (Optics Letters, 24:531-533, 1999), the entire disclosure of which is incorporated herein by reference. In the interferometer, there is a circulator where the emitted light is split to the sample arm and the reference arm. The system also includes a circulator that directs light to the sample and receives reflected light from the sample and directs it toward a detector. The system also includes a circulator that directs light to the reference surface and received reflected light from the reference surface and directs it toward the detector. There is also a circulator at the point at which reflected light from the sample and reflected light from the reference are recombined and directed to the detector.

In a common beam path system, rather than splitting a portion of the light to a reference arm, all of the produced light travels through a single optical fiber. Within the single fiber is a reflecting surface. A portion of the light is reflected off that surface prior to reaching a target tissue (reference) and a remaining portion of the light passes through the reflecting surface and reaches the target tissue. The reflected light from the tissue recombines with the signal from the reference forming interference fringes allowing precise depth-resolved imaging of the target tissue on a micron scale. Common beam path interferometers are further described for example in Vakhtin, et al. (Applied Optics, 42(34):6953-6958, 2003), Wang et al. (U.S. Pat. No. 7,999,938), Tearney et al. (U.S. Pat. No. 7,995,210), and Galle et al. (U.S. Pat. No. 7,787,127), the content of each of which is incorporated by reference herein in its entirety.

The common beam path optical layout of the interferometer includes a single array of optical fibers that are connected to a circulator. The array of optical fibers are configured to accommodate and couple to a catheter. The circulator directs light transmitted from the light source through the array of optical fibers of the common beam path optical layout to a sample and reference, and receives the reflected light from the sample and reference and directs it to the detector.

OCT systems of the invention include a detector. The detector includes photodetection electronics. The detector can support both balanced and non-balanced detection. OCT detectors are described for example in Kemp (U.S. Pat. No. 8,049,900), Kemp (U.S. Pat. No. 7,929,148), Milner (U.S. Pat. No. 7,853,316), Feldman et al. (U.S. Pat. No. 7,711,413), Kemp et al., U.S. Patent Application Publication No. 2012/0224751), Milner et al. (U.S. Patent Application Publication No. 2012/0136259), Kemp et al., (U.S. Patent Application Publication No. 2012/0013914), Milner et al. (U.S. Patent Application Publication No. 2011/0152771), and Kemp et al. (U.S. Patent Application Publication No. 2009/0046295), the content of each of which is incorporated by reference in its entirety.

OCT systems of the invention may conduct any form of OCT known in the art. One manner for conducting OCT may be Swept-Source OCT (“SS-OCT”). SS-OCT time-encodes the wavenumber (or optical frequency) by rapidly tuning a narrowband light source over a broad optical bandwidth. The high speed tunable laser sources for SS-OCT exhibit a nonlinear or non-uniform wavenumber vs. time [k(t)] characteristic. As such, SS-OCT interferograms sampled uniformly in time [S(t), e.g., using an internal digitizer clock] must be remapped to S(k) before Fourier transforming into the path length (z) domain used to generate the OCT image. An SS-OCT system and methods for its use are described in Kemp et al., (U.S. Patent Application Publication No. 2012/0013914). The content of which is incorporated by reference herein in its entirety.

In other embodiments, the imaging device is an IVUS device. The device can be a phased-array IVUS device or a pull-back type IVUS device, including rotational IVUS imaging devices. IVUS imaging devices and processing of IVUS data are described for example in Yock, U.S. Pat. Nos. 4,794,931, 5,000,185, and U.S. Pat. No. 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et al., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et al., U.S. Pat. No. 5,183,048, Eberle et al., U.S. Pat. No. 5,167,233, Eberle et al., U.S. Pat. No. 4,917,097, Eberle et al., U.S. Pat. No. 5,135,486, and other references well known in the art relating to intraluminal ultrasound devices and modalities. All of these references are incorporated by reference herein in their entirety.

IVUS imaging is widely used in interventional cardiology as a diagnostic tool for assessing a diseased vessel, such as an artery, within the human body to determine the need for treatment, to guide an intervention, and/or to assess its effectiveness. An IVUS device including one or more ultrasound transducers is introduced into the vessel and guided to the area to be imaged. The transducers emit and then receive backscattered ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a 360 degree cross-sectional image of the vessel where the device is placed.

There are two general types of IVUS devices in use today: rotational and solid-state (also known as synthetic aperture phased array). For a typical rotational IVUS device, a single ultrasound transducer element is located at the tip of a flexible driveshaft that spins inside a plastic sheath inserted into the vessel of interest. The transducer element is oriented such that the ultrasound beam propagates generally perpendicular to the axis of the device. The fluid-filled sheath protects the vessel tissue from the spinning transducer and driveshaft while permitting ultrasound signals to propagate from the transducer into the tissue and back. As the driveshaft rotates, the transducer is periodically excited with a high voltage pulse to emit a short burst of ultrasound. The same transducer then listens for the returning echoes reflected from various tissue structures. The IVUS imaging system assembles a two dimensional display of the vessel cross-section from a sequence of pulse/acquisition cycles occurring during a single revolution of the transducer. Suitable rotational IVUS catheters include, for example the REVOLUTION 45 MHz catheter (offered by the Volcano Corporation).

In contrast, solid-state IVUS devices carry a transducer complex that includes an array of ultrasound transducers distributed around the circumference of the device connected to a set of transducer controllers. The transducer controllers select transducer sets for transmitting an ultrasound pulse and for receiving the echo signal. By stepping through a sequence of transmit-receive sets, the solid-state IVUS system can synthesize the effect of a mechanically scanned transducer element but without moving parts. The same transducer elements can be used to acquire different types of intravascular data. The different types of intravascular data are acquired based on different manners of operation of the transducer elements. The solid-state scanner can be wired directly to the imaging system with a simple electrical cable and a standard detachable electrical connector.

The transducer subassembly can include either a single transducer or an array. The transducer elements can be used to acquire different types of intravascular data, such as flow data, motion data and structural image data. For example, the different types of intravascular data are acquired based on different manners of operation of the transducer elements. For example, in a gray-scale imaging mode, the transducer elements transmit in a certain sequence one gray-scale IVUS image. Methods for constructing IVUS images are well-known in the art, and are described, for example in Hancock et al. (U.S. Pat. No. 8,187,191), Nair et al. (U.S. Pat. No. 7,074,188), and Vince et al. (U.S. Pat. No. 6,200,268), the content of each of which is incorporated by reference herein in its entirety. In flow imaging mode, the transducer elements are operated in a different way to collect the information on the motion or flow. This process enables one image (or frame) of flow data to be acquired. The particular methods and processes for acquiring different types of intravascular data, including operation of the transducer elements in the different modes (e.g., gray-scale imaging mode, flow imaging mode, etc.) consistent with the present invention are further described in U.S. patent application Ser. No. 14/037,683, the content of which is incorporated by reference herein in its entirety.

The acquisition of each flow frame of data is interlaced with an IVUS gray scale frame of data. Operating an IVUS catheter to acquire flow data and constructing images of that data is further described in O'Donnell et al. (U.S. Pat. No. 5,921,931), U.S. Provisional Patent Application No. 61/587,834, and U.S. Provisional Patent Application No. 61/646,080, the content of each of which is incorporated by reference herein its entirety. Commercially available fluid flow display software for operating an IVUS catheter in flow mode and displaying flow data is CHROMAFLOW (IVUS fluid flow display software offered by the Volcano Corporation). Suitable phased-array imaging catheters include Volcano Corporation's EAGLE EYE Platinum Catheter, EAGLE EYE Platinum Short-Tip Catheter, and EAGLEEYE Gold Catheter.

Besides intravascular ultrasound, other types of ultrasound catheters can be made using the teachings provided herein. By way of example and not limitation, other suitable types of catheters include non-intravascular intraluminal ultrasound catheters, intracardiac echo catheters, laparoscopic, and interstitial catheters. In addition, the probe may be used in any suitable anatomy, including, but not limited to, coronary, carotid, neuro, peripheral, or venous.

### Sensing Device

Any sensing device that is capable of detecting a conformational shape of the imaging device may be used with apparatuses and devices of the invention. Exemplary sensing devices are shown for example in, Jones et al. (U.S. Pat. No. 6,275,628), Murphy (U.S. Pat. No. 6,343,168), Murphy (U.S. Pat. No. 6,021,240), Murphy (U.S. Pat. No. 6,366,722), Murphy et al. (U.S. Pat. No. 5,301,001), Pulliam et al. (U.S. Pat. No. 6,426,796), Meller et al. (U.S. Pat. No. 6,429,421), Wavering et al. (U.S. Pat. No. 6,671,055), Averett et al. (U.S. Pat. No. 6,898,337), Froggatt et al. (U.S. Pat. No. 7,440,087), Froggatt et al. (U.S. Pat. No. 7,515,276), and Childers et al. (U.S. Pat. No. 7,781,724), the content of each of which is incorporated by reference herein in its entirety.

The fiber optic position and shape sensing device of the present invention generally includes an optical fiber for determining position and shape of an object. The optical fiber includes at least two fiber cores spaced apart from each other in which mode coupling between the fiber cores is minimized. The device further includes an array of fiber Bragg gratings disposed within each fiber core and a frequency domain reflectometer positioned in an operable relationship to the optical fiber. The optical fiber is either at least two single core optical fibers positioned in a relative relationship to one another or a multicore optical fiber having at least two fiber cores.

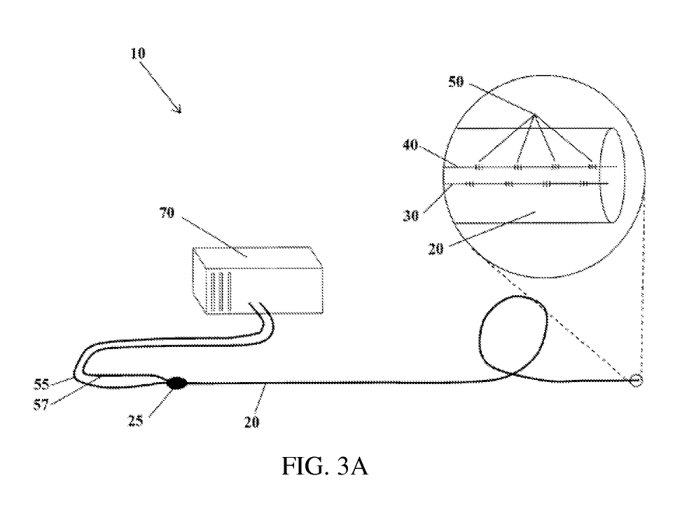

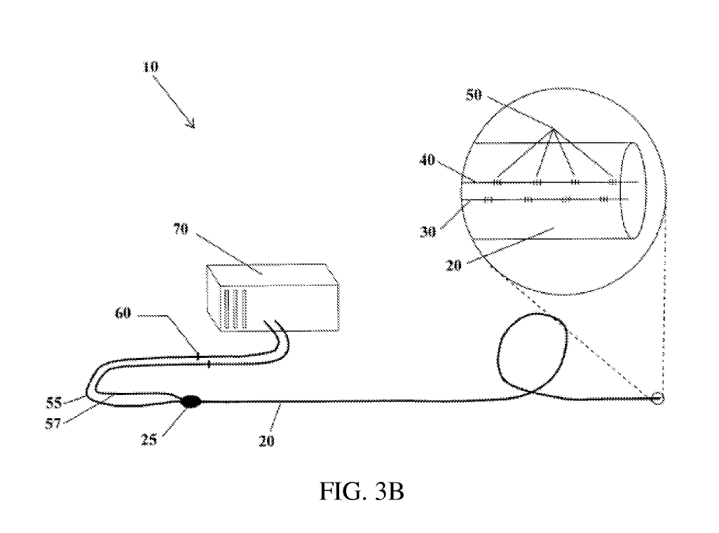

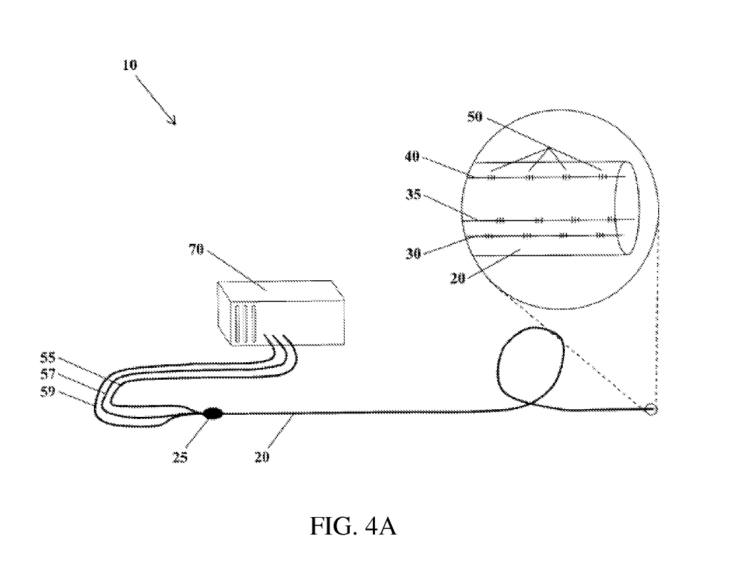

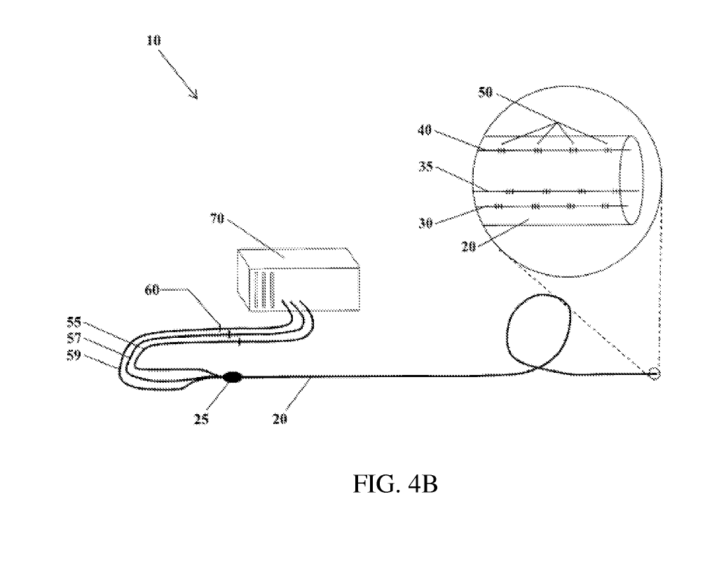

Referring now to the figures where similar elements are numbered the same throughout, FIG. 3A depicts an embodiment of the fiber optic position and shape sensing device 10 of the present invention where the optical fiber is a multicore optical fiber 20 having at least two fiber cores 30, 40 spaced apart in which mode coupling between the fiber cores is minimized. In order to achieve optimal results, mode coupling between the fiber cores should be minimized if not completely eliminated. A multicore optical fiber having two fiber cores (as depicted in FIG. 3) is suitable for use as a positioning device or for determining the two dimensional shape of an object. However, when determining three dimensional shapes, the multicore optical fiber should have preferably three fiber cores 30, 35, 40 (as shown in FIG. 4A).

Multicore optical fiber is fabricated in much the same way as a standard telecommunications optical fiber. The first step in the fabrication process is to design and model the optical parameters for the preform (i.e.—refractive index profile, core/cladding diameters, etc.) to obtain the desired wave guiding performance. The fabrication of multicore optical fiber requires the modification of standard over-cladding and fiberization processes. Though numerous methods can be employed to achieve the desired geometry, the preferred methods are the multi-chuck over-cladding procedure and the stack-and-draw process. In both techniques, the original preforms with the desired dopants and numerical aperture are fabricated via the Modified Chemical Vapor Deposition (MCVD) process. The preforms are then stretched to the appropriate diameters.

Following the preform stretch, the preforms are sectioned to the appropriate lengths and inserted into a silica tube with the other glass rods to fill the voids in the tube. The variation in the two procedures arises in the method in which the preform rods are inserted into the tube. In the multi-chuck method the bait rods and preforms are positioned in the tube on a glass working lathe. A double chuck is used to align the preforms in the tube. Once positioned, the tube is collapsed on the glass rods to form the preform. The preform is then fiberized in the draw tower by a standard procedure known to those of ordinary skill in the art. In the stack-and-draw process, the preforms and the bait rods are positioned together in the silica tube, with the interstitial space filled with additional glass rods. The glass assembly is then drawn into fiber with the appropriate dimensions.

An array of fiber Bragg gratings 50 is disposed within each fiber core. Such array is defined as a plurality of fiber Bragg gratings disposed along a single fiber core. Preferably, the array comprises at least one hundred (100) fiber Bragg gratings. Each fiber Bragg grating is used to measure strain on the multicore optical fiber. Fiber Bragg gratings are fabricated by exposing photosensitive fiber to a pattern of pulsed ultraviolet light from an excimer laser, forming a periodic change in the refractive index of the core. This pattern, or grating, reflects a very narrow frequency band of light that is dependent upon the modulation period formed in the core. In its most basic operation as a sensor, a Bragg grating is either stretched or compressed by an external stimulus. This results in a change in the modulation period of the grating which, in turn, causes a shift in the frequency reflected by the grating. By measuring the shift in frequency, one can determine the magnitude of the external stimulus applied.

Referring back to FIG. 3A, the multicore optical fiber 20 is coupled to single core optical fibers 55, 57 through a coupling device 25. FIG. 4A shows an embodiment of the invention where three single core optical fibers 55, 57, 59 are coupled to the multicore optical fiber 20 through a coupling device 25. FIGS. 3B and 4B depict a preferred embodiment where each single core optical fiber 55, 57 (in FIG. 3B) or 55, 57, 59 (in FIG. 4B) has a broadband reference reflector 60 positioned in an operable relationship to each fiber Bragg grating array in which an optical path length is established for each reflector/grating relationship. However, it is important to note that the broadband reference reflector is not necessary in order for the invention to work. Alternatively, it is well understood in the art that all optical frequency domain reflectometers include a means, such as a reflector, to establish a reference path and, therefore, a separate reflector such as the broadband reference reflector is not an essential element of the invention. Similarly, some optical frequency domain reflectometers (such as the OBR commercially available from Luna Innovations Incorporated) rely on an internal reference path, thus eliminating the need for an external broadband reference reflector altogether. As a preferred embodiment, a frequency domain reflectometer 70 is positioned in an operable relationship to the multicore optical fiber 20 through the single core optical fibers 55, 57, 59 such that the frequency domain reflectometer 70 is capable of receiving signals from the fiber Bragg gratings. As stated previously, any frequency domain reflectometer known to those of ordinary skill in the art may be employed for the present invention provided that it is capable of monitoring many Bragg gratings at one time. Preferably, the frequency domain reflectometer receives signals from the fiber Bragg grating arrays. Such a device is known as the Luna Distributed Sensing System and is commercially available from Luna Innovations Incorporated.

In further embodiments of the invention, the array of fiber Bragg gratings are co-located along the multicore optical fiber. The array preferably includes at least one hundred (100) fiber Bragg gratings. In an alternative embodiment, a wavelength division multiplexing device is positioned in an operable relationship to the multicore optical fiber and is co-located with the frequency domain reflectometer. This arrangement allows for extension of optical fiber length if needed for a specific application, where a much smaller number (less than about one hundred (100) fiber Bragg gratings) are employed.

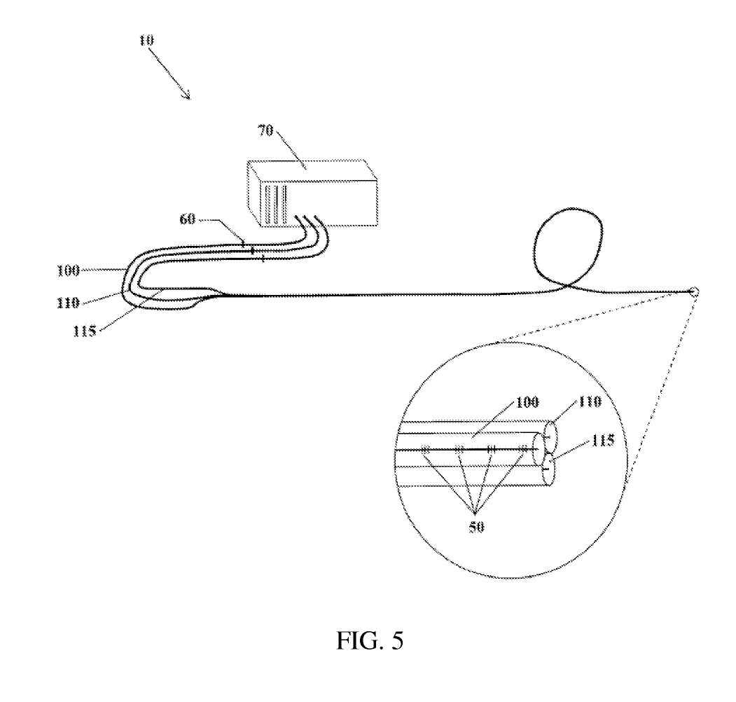

FIG. 5 depicts an alternative preferred embodiment where the optical fiber means is at least two single core optical fibers and, preferably, is three single core optical fibers 100, 110, 115. When three single core optical fibers are used, the fiber cores are non-coplanar and form a triangular shape. In certain embodiments, the triangular shape is such that each fiber core has a center, and each center is 120° with respect to each of the other two core centers. The 120° relationship minimizes distortions. As with the multicore optical fiber, the fiber cores are spaced apart such that mode coupling between the fiber cores is minimized. Also, as seen in the multicore optical fiber, an array of Bragg gratings 50 is disposed within each fiber core. As a preferred embodiment, a broadband reference reflector 60 is positioned in an operable relationship to each fiber Bragg grating array wherein an optical path length is established for each reflector/grating relationship, however, the broadband reference reflector 60 is not essential. A frequency domain reflectometer 70 is positioned in an operable relationship to the single core optical fibers.

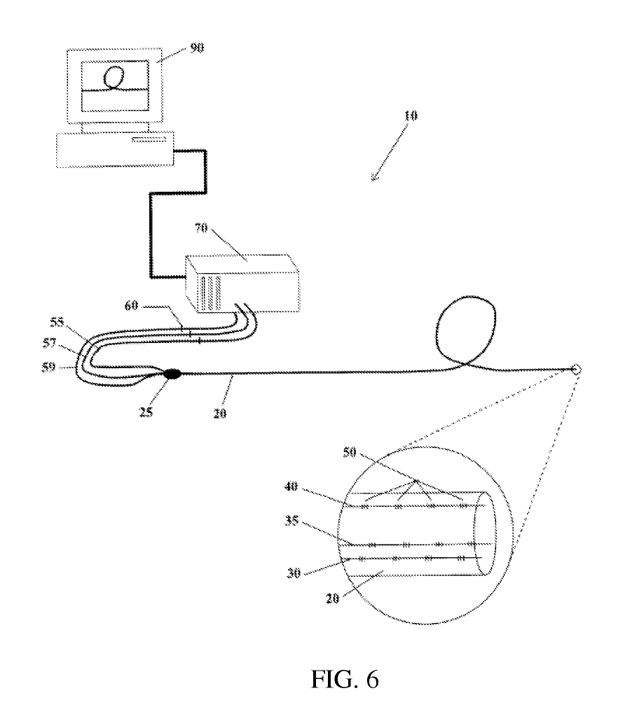

In a further embodiment of the invention, shown in FIG. 6, the fiber optic position and shape sensing device 10 has a computer 90 positioned in an operable relationship to the frequency domain reflectometer 70. It is understood that the optical arrangement shown in FIG. 6 is not limited to those devices employing multicore optical fibers but that it may be used in combination with those devices employing single core optical fibers as well. The computer correlates the signals received from the frequency domain reflectometer 70 to strain measurements. These strain measurements are correlated into local bend measurements. A local bend measurement is defined as the bend between a reference sensor and the next set of sensors in the array. The local bend measurements are integrated into a position or shape. If the optical fiber means has only two cores, then shape determination is limited to two dimensions, if there are three or more cores, three dimensional shape is determined, and in both instances, position is determined.

In essence, the present invention operates on the concept of determining the shape of an object by measuring the shape of the optical fiber. Based on these measurements relative position is also ascertainable. For example, shape sensing is accomplished by creating a linear array of high spatial resolution fiber optic bend sensors. Assuming each element is sufficiently small, by knowing the curvature of the structure at each individual element the overall shape is reconstructed through an integration process. A bend sensor is created by adhering two strain sensors to either side of a flexible object or by embedding the sensors in the object. An exemplary device is a catheter. To monitor the shape of an object that can deform in three dimensions, a measure of the full vector strain is required. Hence, a minimum of three cores is required with each core containing an array of fiber Bragg grating strain sensors (preferably of at least one hundred (100) fiber Bragg gratings), preferably each sensor collocated in the axial dimension. To form an array of three dimensional bend sensors, it is assumed that, at a minimum, three optical fiber cores are fixed together such that their centers are non-coplanar. Preferably, the core centers are each 120° with respect to each of the other two core centers and form a triangular shape. It should be acknowledged that any number of optical fiber cores greater than three can also be used for three dimensional bend sensing. The separate cores of the optical fiber containing the fiber Bragg grating strain sensor arrays are embedded into a monolithic structure. By co-locating these strain sensors down the length of the structure whereby sensing points are created, the differential strain between the cores is used to calculate curvature along the length of the structure. By knowing the curvature of the structure at each individual sensing point the overall shape of the structure is reconstructed, presuming that each individual sensing point is sufficiently small.

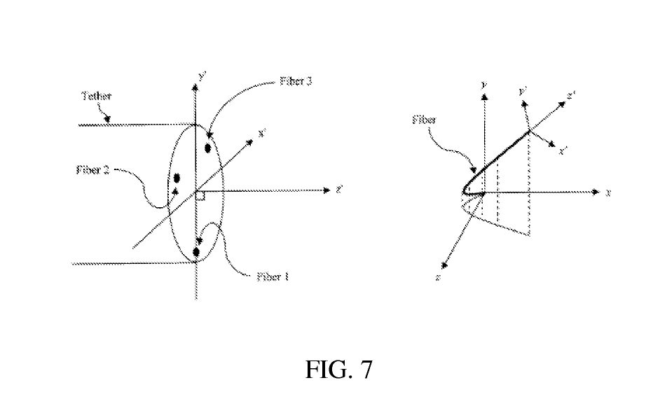

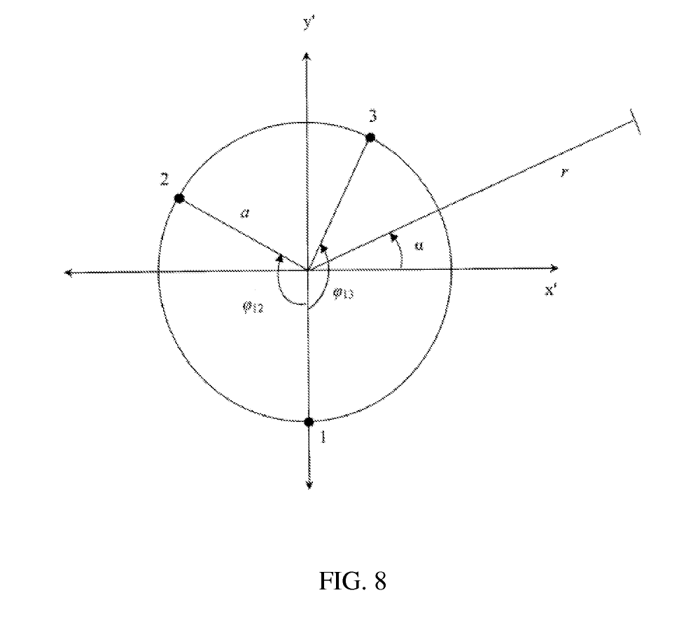

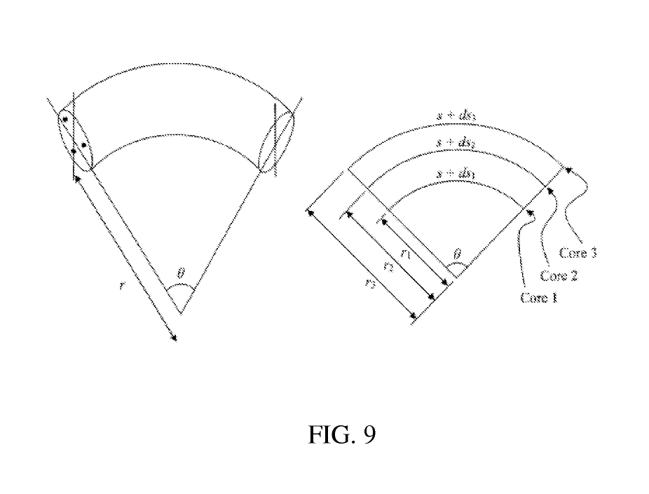

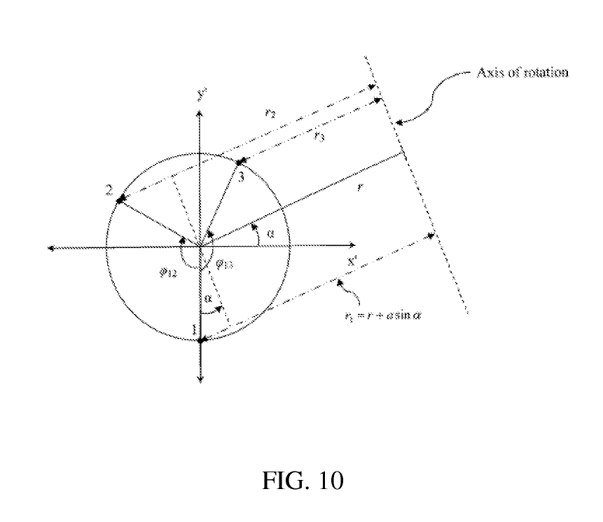

Strain values for each segment of an object (such as a tether) are used to compute a bend angle and bend radius for each segment of the object. Starting from the beginning of the object, this data is then used to compute the location of the next sensor triplet along the object and to define a new local coordinate system. An algorithm interpolates circular arcs between each sensor triplet on the object. The geometry of the remainder of the object is determined by repeating the process for each sensor triplet along the length of the object. Since the fiber Bragg gratings in each sensing fiber are collocated, a triplet of strain values at evenly spaced segments along the object exists. For each step along the object, a local coordinate system (x′, y′, z′) is defined called the sensor frame. This coordinate system has its origin at the center of the object's perimeter for any given sensor triplet. The z′ axis points in the direction of the object and the y′ axis is aligned with fiber 1 (see FIG. 7). Using the three strain values (ε₁, ε₂, ε₃) for a given sensor triplet one can calculate the direction of the bend, α, with respect to the x′ axis as well as the bend radius, r, which is the distance from the center of curvature to the center of the core perimeter (see FIG. 8). Knowing r and α for a particular object segment permits the computation of the coordinates of the end of the segment in the (x′, y′, z′) coordinate system. The beginning of the fiber segment is taken to be the origin of the (x′, y′, z′) system. When there is no curvature to the fiber segment, each core segment has a length s. When a curvature is introduced each core is generally a different distance (r₁, r₂, r₃) from the center of curvature, as shown in FIG. 9. Since all of the core segments subtend the same curvature angle, θ, each segment must have a different length. The change in length due to bending the fiber is denoted as ds₁, ds₂ and ds₃ as shown in FIG. 9.

From the geometry shown in FIG. 9, the equations relating the change in length and radius of curvature of each fiber to the other fibers are derived as:

$$θ = \frac{s + {ds}_{1}}{{r}_{1}} = \frac{s + {ds}_{2}}{{r}_{2}} = \frac{s + {ds}_{3}}{{r}_{3}} \qquad ( 1 )$$

Since strain (denoted by ε) is defined as the ratio of the change in length of the fiber, ds to its unstretched length s (i.e. ε=ds/s) the first part of Equation 1 is written in terms of the measured strains.

$$θ = \frac{s + {ds}_{1}}{{r}_{1}} = s ( \frac{1 + {ds}_{1} / s}{{r}_{1}} ) = s ( \frac{1 + {ɛ}_{1}}{{r}_{1}} ) \qquad ( 2 )$$

Extending this argument to the other terms of Equation 1 the following expression results:

$$\frac{1 + {ɛ}_{1}}{{r}_{1}} = \frac{1 + {ɛ}_{2}}{{r}_{2}} = \frac{1 + {ɛ}_{3}}{{r}_{3}} \qquad ( 3 )$$

In order to solve Equation 3 for r and α, r₁, r₂, and r₃ need to be written in terms of r and α. This can be done by analyzing the geometry of the fiber cross-section (FIG. 10) and results in the following expressions for the radii of curvature for each of the fibers:

r₁=r+α sin α

r₂=r+α sin(α+φ₁₂)

r₂=r+α sin(α−φ₁₃) (4)

Using Equations 4 to make substitutions in Equations 3 the following three equations are derived for r and α. These equations are:

(1+ε₁)(r+α sin(α+φ₁₂))=(1+ε₂)(r+α sin(α))

(1+ε₁)(r+α sin(α−φ₁₃))=(1+ε₃)(r+α sin(α))

(1+ε₂)(r+α sin(α−φ₁₃))=(1+ε₃)(r+α sin(α+φ₁₂)) (5)

In order to make these equations easier to follow the following substitutions are made.

ε₁₂=ε₂−ε₁ ε₁₃=ε₃−ε₁ ε₂₃=ε₃−ε₂

σ₁=1+ε₁ σ₂=1+ε₂ σ₃=1+ε₃ (6)

After a bit of algebra the following solution is found for α.

$$tan α = \frac{{ɛ}_{13} sin {ϕ}_{12} + {ɛ}_{12} sin {ϕ}_{13}}{{ɛ}_{23} - {ɛ}_{13} cos {ϕ}_{12} + {ɛ}_{12} cos {ϕ}_{13}} \qquad ( 7 )$$

It is clear from Equation 7 that the bend angle is dependent only on the differential strains, not the absolute strain values. The bend radius r can be computed in three different ways. Each of these formulae give the same solution for r but it is useful during implementation to have at least two handy in case one of the differential strains (defined in Equations 6) turns out to be zero.

$$r = \{ \begin{matrix}\frac{a}{{ɛ}_{12}} ( {σ}_{1} sin ( α + {ϕ}_{12} ) - {σ}_{2} sin ( α ) ) \\ \frac{a}{{ɛ}_{13}} ( {σ}_{1} sin ( α - {ϕ}_{13} ) - {σ}_{3} sin ( α ) ) \\ \frac{a}{{ɛ}_{23}} ( {σ}_{2} sin ( α - {ϕ}_{13} ) - {σ}_{3} sin ( α + {ϕ}_{12} ) )\end{matrix}$$

Clearly, Equation 7 shows that −π/2<α<π/2. The extra π radians appear in the r calculation. That is, if r is negative, simply negate r and add π to α. After this operation, r>0 and 0≦α<2π. Also, when implementing an algorithm, cases where ε₁=ε₂=ε₃ form a special case where the bend angle is arbitrary because the bend radius is infinite (zero curvature).

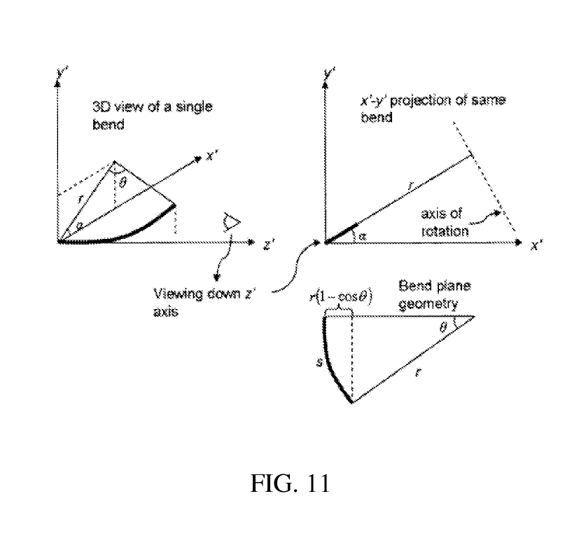

SENSOR FRAME POSITION CALCULATION. Knowing r and a for α particular tether segment permits the computation of the coordinates of the end of the segment in the (x′, y′, z′) coordinate system. The beginning of the fiber segment is taken to be the origin of the (x′, y′, z′) system. From FIG. 11 the relationship between (r, α) and the endpoint of the segment (x′, y′, z′) can be derived. The angle θ, shown in the diagram is related to the bend radius, r, and the tether segment length, s, through θ=s/r.

From FIG. 11 it is not difficult to see that the proper expressions for the coordinates of the end of the tether are given by

x′=r(1−cos θ)cos α

y′=r(1−cos θ)sin α

z′=r sin θ (9)

LABORATORY FRAME POSITION CALCULATION. In order to reference the coordinates (x′, y′, z′) back to the laboratory frame of reference (x, y, z), the orientation of the \hat{x}, ŷ, \hat{z}, basis vectors with respect to the lab frame for each segment of the tether must be tracked. In order to accomplish this, a rotation matrix is utilized that will rotate a vector through an angle θ about an axis of rotation. This rotation is performed with the rotation matrix, R, defined as follows:

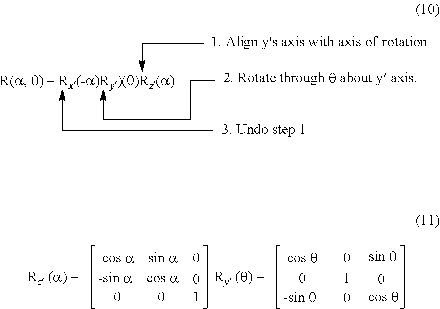

The transformation matrix R, after multiplying the three rotation matrices is:

$$( 12 )$$

$$R ( α , θ ) = [ \begin{matrix}cos ( θ ) {cos}^{2} ( α ) + {sin}^{2} ( α ) & ( cos θ - 1 ) sin ( α ) cos ( α ) & sin ( θ ) cos ( α ) \\ ( cos ( θ ) - 1 ) sin ( α ) cos ( α ) & cos ( θ ) {sin}^{2} ( α ) + {cos}^{2} ( α ) & sin ( θ ) sin ( α ) \\ - sin ( θ ) cos ( α ) & - sin ( θ ) sin ( α ) & cos ( θ )\end{matrix} ] .$$

The rotation matrix R is used to translate the basis vectors from one segment of the tether into basis vectors for the next segment in terms of the first segment's basis vectors. The subscript ‘n’ signifies variables related to the n^{th} tether segment. The notation Rₙ^{ij} refers to particular elements (row i, column j) of the rotation matrix R(αₙ, θₙ) given in equation 12 for the n^{th} sensor triplet. Also, the subscript n on the column vectors denotes that these vectors are referenced to the \hat{x}_{n′}, ŷ_{n′}, \hat{z}_{n′}) basis.

$$\begin{matrix}{\hat{x}}_{n + 1}^{′} = {R ( {α}_{n} , {θ}_{n} ) [ \begin{matrix}1 \\ 0 \\ 0\end{matrix} ]}_{n} \\ = {[ \begin{matrix}{R}_{n}^{11} \\ {R}_{n}^{21} \\ {R}_{n}^{31}\end{matrix} ]}_{n} \\ = {R}_{n}^{11} {\hat{x}}_{n}^{′} + {R}_{n}^{21} {\hat{y}}_{n}^{′} + {R}_{n}^{31} {\hat{z}}_{n}^{′}\end{matrix} \\ \begin{matrix}{\hat{y}}_{n + 1}^{′} = {R ( {α}_{n} , {θ}_{n} ) [ \begin{matrix}0 \\ 1 \\ 0\end{matrix} ]}_{n} \\ = {[ \begin{matrix}{R}_{n}^{12} \\ {R}_{n}^{22} \\ {R}_{n}^{32}\end{matrix} ]}_{n} \\ = {R}_{n}^{12} {\hat{x}}_{n}^{′} + {R}_{n}^{22} {\hat{y}}_{n}^{′} + {R}_{n}^{32} {\hat{z}}_{n}^{′}\end{matrix} \\ \begin{matrix}{\hat{z}}_{n + 1}^{′} = {R ( {α}_{n} , {θ}_{n} ) [ \begin{matrix}0 \\ 0 \\ 1\end{matrix} ]}_{n} \\ = {[ \begin{matrix}{R}_{n}^{13} \\ {R}_{n}^{23} \\ {R}_{n}^{33}\end{matrix} ]}_{n} \\ = {R}_{n}^{13} {\hat{x}}_{n}^{′} + {R}_{n}^{23} {\hat{y}}_{n}^{′} + {R}_{n}^{33} {\hat{z}}_{n}^{′}\end{matrix} \qquad ( 13 )$$

Equations 13 represent a recursion relation for the orientation of any given segment of the sensing fiber. If the prime coordinate system initially coincides with the lab system:

\hat{x}₁′=\hat{x}ŷ₁′=ŷ\hat{z}₁′=\hat{z} (14)

Starting with the basis vectors given in equations 14, equations 13 can then be used to compute the basis vectors at any point along the tether in terms of the laboratory basis vectors (\hat{x}, ŷ, \hat{z}). That is, the sensor frame basis vectors for any sensor triplet along the tether may be written in the following form:

\hat{x}ₙ′=cₙ¹¹\hat{x}+cₙ¹²ŷ+cₙ¹³\hat{z}

ŷₙ′=cₙ²¹\hat{x}+cₙ²²ŷ+cₙ²³\hat{z}

\hat{z}ₙ′=cₙ³¹\hat{x}+cₙ³²ŷ+cₙ³³\hat{z} (15)

By using the general expressions of equations 15 in conjunction with the recursion relations in equations 12, a general expression for the constants cₙ^{ij} in equation 15 can be written. First consider the term cₙ₊₁¹¹. It is known from equations 13 that

\hat{x}ₙ₊₁′=Rₙ¹¹\hat{x}ₙ′+Rₙ²¹ŷₙ′+Rₙ³¹\hat{z}ₙ′. (16)

Therefore the component of \hat{x}_{n+1′} in the \hat{x} direction (i.e. cₙ₊₁¹¹) must include any components of \hat{x}_{n′}, ŷ_{n′}, and \hat{z}_{n′} and that are in the \hat{x} direction. These components are shown in equations 15. Therefore the following expression for cₙ₊₁¹¹ can be derived:

cₙ₊₁¹¹=Rₙ¹¹cₙ¹¹+Rₙ²¹cₙ²¹+Rₙ³¹cₙ³¹ (17)

Following the same procedure for cₙ₊₁¹²:

cₙ₊₁¹²=Rₙ¹¹cₙ¹²+Rₙ²¹cₙ²²+Rₙ³¹cₙ³². (18)

Now a general recursion relation for cₙ₊₁^{ij} can be shown to be:

$${c}_{n + 1}^{ij} = {\sum }_{k = 1}^{3} {R}_{n}^{ki} {c}_{n}^{kj} \qquad ( 19 )$$

From equations 14 it can be seen that the initial conditions for this relation are given by:

$${c}_{1}^{ij} = [ \begin{matrix}1 & 0 & 0 \\ 0 & 1 & 0 \\ 0 & 0 & 1\end{matrix} ] . \qquad ( 20 )$$

The necessary information to construct the relative position of the tether in the laboratory frame now exists. For the n^{th} tether segment, the displacement vector, wₙ, from start to end is given by the expression:

wₙ=xₙ′\hat{x}ₙ′+yₙ′ŷₙ′+zₙ′\hat{z}ₙ′. (21)

The components of wₙ are given by equations 9, while the basis vectors can be referenced to the lab frame via equations 15 & 19. Having wₙ in the lab basis vectors (\hat{x}, ŷ, \hat{z}), an expression for the position, sₙ, of the end of the n^{th} segment of the tether in the lab frame is then derived to be:

$${s}_{n} = {\sum }_{m = 1}^{n} {w}_{m} . \qquad ( 22 )$$

The previous sections described a method for finding the location of the center point on the tether from strain data for collocated sensors. In order to implement this algorithm in software it seems that a good approach is to take advantage of the recursion relations that were developed by starting at the beginning of the tether (where the lab and sensor frames coincide) and computing the endpoint of each tether in the laboratory frame sequentially. Previously, the equations necessary to translate the strains from each sensor triplet along the tether into a list of bend radii rₙ and angles αₙ as well as the position of the end of a tether segment in the sensor frame of reference were derived. Generalizing these results to the n^{th} tether segment yields:

xₙ′=rₙ(1−cos θₙ)cos αₙ

yₙ′=rₙ(1−cos θₙ)sin αₙ,

zₙ′=rₙ sin θₙ (23)

where θₙ=s/rₙ. In addition, the relationship between the sensor frame coordinates and the laboratory frame coordinates was derived. Using these relationships, a recursive relationship for the position of the end of any tether segment in the laboratory frame can be derived as follows:

$${s}_{n} = {\sum }_{m = 1}^{n} {w}_{m} = {\sum }_{m = 1}^{n} {x}_{m}^{′} {\hat{x}}_{m}^{′} + {y}_{m}^{′} + {z}_{m}^{′} {{z}^{□}}_{m}^{′} , \qquad ( 24 )$$

where the basis vectors are referenced to the laboratory reference frame by:

$$\begin{matrix}{\hat{x}}_{n}^{′} = {c}_{n}^{11} \hat{x} + {c}_{n}^{12} \hat{y} + {c}_{n}^{13} \hat{z} \\ {\hat{y}}_{n}^{′} = {c}_{n}^{21} \hat{x} + {c}_{n}^{22} \hat{y} + {c}_{n}^{23} \hat{z} , \\ {\hat{z}}_{n}^{′} = {c}_{n}^{31} \hat{x} + {c}_{n}^{32} \hat{y} + {c}_{n}^{33} \hat{z} \\ and & ( 25 ) \\ {c}_{n + 1}^{ij} = {\sum }_{k = 1}^{3} {R}_{n}^{ki} {c}_{n}^{kj} , & ( 26 )\end{matrix}$$

where Rₙ^{ij} is given by:

$${R}_{n}^{ij} = [ \begin{matrix}cos ( {θ}_{n} ) {cos}^{2} ( {α}_{n} ) + {sin}^{2} ( {α}_{n} ) & ( cos {θ}_{n} - 1 ) sin ( {α}_{n} ) cos ( {α}_{n} ) & sin ( {θ}_{n} ) cos ( {α}_{n} ) \\ ( cos ( {θ}_{n} ) - 1 ) sin ( {α}_{n} ) cos ( {α}_{n} ) & cos ( {θ}_{n} ) {sin}^{2} ( {α}_{n} ) + {cos}^{2} ( {α}_{n} ) & sin ( {θ}_{n} ) sin ( {α}_{n} ) \\ - sin ( {θ}_{n} ) cos ( {α}_{n} ) & - sin ( {θ}_{n} ) sin ( {α}_{n} ) & cos ( {θ}_{n} )\end{matrix} ] , \qquad ( 27 )$$

with the initial condition

$${c}_{1}^{ij} = [ \begin{matrix}1 & 0 & 0 \\ 0 & 1 & 1 \\ 0 & 0 & 1\end{matrix} ] . \qquad ( 28 )$$

Therefore, equations 23-28 in conjunction with the solutions for α and r given in equations 7 and 8 form the set of results necessary to translate measured strain data into three dimensional position data.

### Combination of OCT Device and Sensing Device

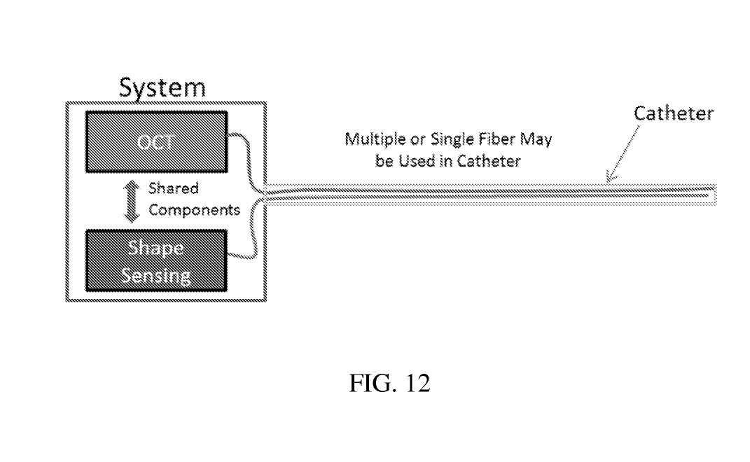

In certain embodiments, the invention provides a single apparatus that includes an OCT device and a sensing device, for example as described above. FIG. 12 shows an example of such a device, which leverages components that can be shared between the OCT device and sensing device in order to save costs, space, and other valuable resources. In the exemplary device shown in FIG. 12, components that may be shared include the data acquisition (DAQ) circuit, detectors, coherent light source or tunable laser, and, laser controller.

The control system performs several functions including receiving and analyzing digital data outputted by the DAQ circuit and outputting control signals to the laser controller. The control system outputs control signals over a data bus for input to the laser controller. The control system may be implemented by a microprocessor or computer having a random access memory (RAM). The RAM should be large enough to perform signal processing algorithms such as Fourier Transform analysis. The control system may be implemented by any one of the many commercially available personal computers. In certain embodiments, the RAM may have a size of at least 40 megabytes. In certain embodiments, the functions of the control system are implemented by a software program that provides built-in hardware interfaces, displays and signal processing algorithms. In other embodiments, the software program is configured with a programming language such as LABVIEW (data processing software). LABVIEW (data processing software) provides built-in hardware interfaces, displays and signal processing algorithms that significantly reduce the total amount of programming actually required.

The laser controller may be a commercially available external cavity tunable laser controller and includes a piezoelectric tuning (PZT) system. The laser controller generally includes circuitry to provide the drive current, temperature stabilization, picomotor tuning and drive voltage for the PZT system. Positioning the picomotor is accomplished through commands outputted by the control system over the data bus. These commands are inputted into a data input port of the laser controller. The PZT system receives an analog control voltage from the DAQ circuit and tunes the laser to a specific wavelength. The actual wavelength depends upon the magnitude of the analog control voltage. The PZT tunes the laser over a predetermined range of wavelengths in response to a corresponding range of analog control voltages.

The laser controller may be realized by a commercially available laser controller. In certain embodiments, the laser controller has operational characteristics similar to the New Focus 6200 External Cavity Tunable Laser Controller, manufactured by New Focus of Santa Clara, Calif. The New Focus 6200 Controller has a GPIB port for receiving data from the data bus. The New Focus 6200 Controller also includes a PZT system that is able to tune the laser over the range of 0.29 nm (nanometer).

In certain embodiments, the laser is an external cavity laser that is tuned by changing the angle of a Bragg grating within the cavity and is tunable over a predetermined bandwidth. The laser may be realized by a commercially available tunable laser. In certain embodiments, the laser has operational characteristics similar to the New Focus 6213 Laser. The New Focus 6213 Laser is tunable over its 1310 nm gain bandwidth, has a linewidth of about 100 KHz and output power of about 1 mW (milliwatt).

The detectors are optical receivers. Each detector is configured to detect and convert the power or intensity of interference fringes at the detector's input into a voltage. In certain embodiments, each detector has operational characteristics similar to the commercially available New Focus 2011 Front End Optical Receiver.

The DAQ circuit includes an analog-to-digital converter (ADC) and a digital-to-analog converter (DAC). The data bus transfers data between the DAQ circuit and the control system. The ADC circuit converts the outputs of the detectors into a pair of multi-bit signals. Since high resolution is desired, it is important if each multi-bit signal includes at least has sixteen (16) bits. The analog-to-digital conversions are made at a predetermined conversion rate. Preferably, the conversion rate is between about 10 kHz and 20 kHz. The DAC within the DAQ circuit receives a multi-bit signal over the data bus from the control system and converts the multi-bit signal into analog an control voltage. In order to achieve high resolution, the multi-bit signal input into the DAC circuit is comprised of at least twelve (12) bits. As discussed above, the analog control voltage controls the tuning of the PZT system of the controller. The DAQ circuit may be realized by a commercially available data acquisition card. In certain embodiments, the DAQ circuit has operational characteristics similar to the commercially available National Instruments NB-MI-16XH-42 Data Acquisition and Control Card manufactured by National Instruments of Austin, Tex. The NB-MIO-16XH-42 can convert analog signals on two (2) channels at a 12 KHz rate to a pair of sixteen (16) bit signals.

Construction of Image from Imaging and Sensing Devices

As previously described herein, the invention combines imaging devices with three dimensional shape sensing devices in order to produce more accurate images of an inside of a vessel. In certain embodiments, with apparatuses and methods of the invention, the image data (acquired by the imaging device) and the sensing data (acquired by the sensing device) are used in combination with one another to construct a three dimensional image of an inside of the vessel. In particular, the image and sensing data are co-registered with one another to produce images that are more accurate than previously constructed images from intravascular devices alone because constructed images of the invention account for distortions, such as catheter eccentricity or an angle of the catheter inside a lumen.

Co-registration generally refers to any method of re-aligning images, and in particular aligning or overlaying images from different modalities. Co-registration is often used to overlay structural and functional images as well as link functional scans to anatomical scans. Details regarding image co-registration can be found in, for example, in U.S. Pat. Nos. 7,930,104; and 8,298,147; and U.S. patent application Ser. No. 13/388,932, each of which is incorporated herein by reference in its entirety.

An exemplary method of co-registration consistent with the present invention is now described which uses intravascular ultrasound and shape sensing to obtain a co-registered intravascular data set. The invention, however, encompasses any and all intravascular imaging modalities, including without limitation, intravascular ultrasound (IVUS), optical coherence tomography (OCT), external ultrasound, x-ray angiography, Computerized Tomography (CT) angiography, and Magnetic Resonance (MR) angiography. Such modalities can be used instead of intravascular ultrasound and shape sensing modalities and also in addition to such modalities. Any number of modalities is useful for co-registration. Furthermore, modalities suitable for co-registration include functional measurement parameters, in addition, or alternatively, to the shape sensing data, including vessel flow, vessel pressure, FFR, iFR, CFR, etc.

The invention may include a system, including one or more components illustrated and described in FIG. 12, for carrying out the present invention in the form of co-registration of image data and shape sensing data. For example, the system may include one or more processors, memory, input/output, controllers, and the like for carrying out the co-registration methods. In one embodiment, the data acquisition (DAQ) circuit may also function as a co-registration processor, such that it may be configured to carry out one or more functions related to the co-registration process. In other embodiments, a separate co-registration processor (not shown) may be used for carrying out the co-registration of the image and sensing data.

In one embodiment, the DAQ is configured to receive image data from the imaging device and shape sensing data from the sensing device. As previously described, the imaging device, such as an IVUS catheter, can be inserted within a patient so that a diagnostic probe (in particular an IVUS probe) is in the vicinity of a desired imaging location of a vessel. By way of example, the diagnostic probe generates ultrasound waves, receives ultrasound echoes representative of a region proximate the diagnostic probe, and converts the ultrasound echoes to corresponding electrical signals. The corresponding electrical signals are transmitted along the length of the imaging catheter to a proximal connector communicatively coupled to an image processor, which may include, for example, the DAQ. The image processor converts the signals received into cross-sectional images of vessel segments. Additionally, the image processor generates longitudinal cross-sectional images corresponding to slices of a vessel taken along the vessel's length. The image data rendered by the image processor may be initially stored within the processor.

During operation of the imaging device (e.g., acquisition of image data of the vessel), the sensing device is generally configured to detect the conformational shape of the imaging device so as to acquire shape sensing data. As previously described, the sensing device includes an optical fiber having an array of high spatial resolution fiber optic bend sensors positioned thereon. The sensing device may be included within the same catheter of the imaging device, for example. As such, the sensors are configured to monitor the shape of the optical fiber and further determine the shape of the vessel based on strain (or bend) measurements of fiber along a length thereof. Furthermore, the sensing device includes a computing system, for example, configured to translate measured strain data into three dimensional positional data based on a frame of reference (e.g., local coordinate system (x′, y′, z′)) for each sensing event of a segment of the fiber.

A co-registration processor, which may include DAQ, may receive the image data and sensing data and render a co-registration image including both IVUS image and shape sensing data frames derived from the received image data and shape sensing data. During course of a catheterization procedure, the co-registration processor may be configured to receive and store the IVUS image data and the shape sensing data within respective locations of memory. The individually rendered frames of shape sensing data may be appropriately tagged (e.g., time stamp, sequence number, etc.) to correlate IVUS image frames and corresponding shape sensing data frames.

The co-registration processor further renders a co-registration image from the image and shape sensing data stored within the memory. By way of example, a particular IVUS image frame/slice is selected from memory and the co-registration processor identifies shape sensing data from memory corresponding to the selected IVUS image data. The co-registration processor then superimposes or co-aligns the selected IVUS image and shape sensing frames so as to construct a three dimensional image of an inside of the vessel.

FIG. 2 shows an exemplary image constructed from co-registered image data and shape sensing data acquired by the imaging and sensor devices, respectively, of the present invention. As shown, an image produced by co-registering the image and sensing data may be more accurate than previously constructed images from intravascular devices alone because constructed images of the invention account for distortions, such as catheter eccentricity or an angle of the catheter inside a lumen.

Systems and methods for co-registering intravascular data can be found in, for example, U.S. Pat. No. 8,298,147; U.S. Patent Publication. Nos. 2012/0230565; 2011/0319752; and 2013/0030295; and U.S. patent application Ser. No. 13/388,932; 61/776,863, 61/776,858; 61/777,155; 61/777,860; 61/779,610; and 61/792,230, each of which is incorporated herein by reference in its entirety.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. An apparatus for imaging inside a vessel, the apparatus comprising:
an imaging device configured to image an inside of a vessel; and
a sensing device configured to detect a conformational shape of the apparatus.

2. The apparatus according to claim 1, further comprising a hollow body configured to fit within a lumen of a vessel.

3. The apparatus according to claim 2, wherein the imaging device and the sensing device are each at least partially disposed within the hollow body.

4. The apparatus according to claim 3, wherein the imaging device is an optical coherence tomography (OCT) device.

5. The apparatus according to claim 4, wherein the OCT device comprises a light source and at least one optical fiber.

6. The apparatus according to claim 5, wherein the sensing device employs the light source and the optical fiber of the OCT device to detect the conformational shape of the apparatus.

7. The apparatus according to claim 6, wherein the sensing device comprises at least one Fiber Bragg Grating.

8. The apparatus according to claim 4, wherein the OCT device comprises a differential path interferometer.

9. The apparatus according to claim 4, wherein the OCT device comprises a common path interferometer.

10. The apparatus according to claim 4, wherein the OCT device a swept-source OCT device.

11. A method for producing a three dimensional image of a vessel, the method comprising:
using an imaging device to obtain image data of a vessel;
using a sensing device to obtain data about a conformational shape of the imaging device; and
producing a three dimensional image of the vessel based on the image data and the sensing data.

12. The method according to claim 11, wherein the imaging device and the sensing device are combined in a single apparatus.

13. The method according to claim 12, wherein the single apparatus comprising a hollow body configured to fit within a lumen of a vessel.

14. The method according to claim 13, wherein the imaging device and the sensing device are each at least partially disposed within the hollow body.

15. The method according to claim 14, wherein the imaging device is an optical coherence tomography (OCT) device.

16. The method according to claim 15, wherein the OCT device comprises a light source and at least one optical fiber.

17. The method according to claim 16, wherein the sensing device employs the light source and the optical fiber of the OCT device to detect the conformational shape of the imaging device.

18. The method according to claim 17, wherein the sensing device comprises at least one Fiber Bragg Grating.

19. The method according to claim 15, wherein the OCT device comprises a differential path interferometer.

20. The method according to claim 15, wherein the OCT device comprises a common path interferometer.

21. The method according to claim 15, wherein the OCT device a swept-source OCT device.
